(19) United States
(12) Patent Application Publication
(10) Pub. No.: US 2018/0368697 A1
(43) Pub. Date: Dec. 27, 2018

(54) INFORMATION PROCESSING APPARATUS AND SYSTEM

(71) Applicant: CANON KABUSHIKI KAISHA, Tokyo (JP)

(72) Inventors: Kazuhiko Fukutani, Yokohama-shi (JP); Hiroshi Abe, Yokohama-shi (JP); Kazuhito Oka, Tokyo (JP)

(21) Appl. No.: 16/014,109

(22) Filed: Jun. 21, 2018

(30) Foreign Application Priority Data
Jun. 23, 2017 (JP) 2017-123596
May 22, 2018 (JP) 2018-097905

(51) Int. Cl. A61B 5/00; A61B 8/00; G01N 29/24

(52) U.S. Cl. CPC A61B 5/0095; G01N 29/2418; A61B 8/4245

(57) ABSTRACT

An information processing apparatus which processes image data indicating characteristic information at a plurality of positions inside an object based on an acoustic wave generated when the object is irradiated with light via a light irradiating unit, the positional relationship of which with an acoustic probe is defined. The information processing apparatus includes an image data acquiring unit configured to acquire the image data; an information acquiring unit configured to acquire identification information which identifies a type of the acoustic probe; and a correcting unit configured to correct, based on the identification information, an effect on an image value of the image data attributable to a variation in light intensities, at the plurality of positions inside the object, of light irradiated via the light irradiating unit.

### BACKGROUND OF THE INVENTION

### Field of the Invention

The present invention relates to an apparatus for acquiring information on an object using acoustic waves.

### Description of the Related Art

Photoacoustic imaging is known as a technique for imaging functional information of the inside of an object such as structural information and physiological information.

When a living organism that is an object is irradiated with light such as laser light, an acoustic wave (typically, an ultrasonic wave) is generated when the light is absorbed by living tissue inside the object. This phenomenon is referred to as a photoacoustic effect and an acoustic wave generated by the photoacoustic effect is referred to as a photoacoustic wave. Since tissues constituting the object have respectively different absorption rates of light energy, sound pressures of generated photoacoustic waves also differ. With photoacoustic imaging (PAI), by receiving a generated photoacoustic wave with a probe and mathematically analyzing a received signal, characteristic information of the inside of an object can be acquired.

On the other hand, ultrasonic imaging is known as a method of acquiring structural information of the inside of an object. In ultrasonic imaging, ultrasonic waves are transmitted to an object from a plurality of acoustic elements (transducers) arranged on a probe. Subsequently, reflected waves created on interfaces with different acoustic impedances inside the object are received to generate image data.

As a related technique, for example, Japanese Patent Application Laid-open No. 2017-000660 discloses a technique in which an image due to photoacoustic imaging and an image due to ultrasonic imaging are acquired using a same hand-held probe.

Japanese Patent Application Laid-open No. 2017-000660 describes a configuration in which a member (a light irradiating unit) that irradiates an object with light is attachable and detachable to and from a probe and is replaceable. However, changing a combination of a light irradiating unit and a probe causes a positional relationship between acoustic elements and the light irradiating unit and characteristics of light which irradiates the object to change. In addition, there is a problem in that, due to such changes, a light intensity distribution inside the object changes and, accordingly, visibility when imaging object information changes.

### SUMMARY OF THE INVENTION

The present invention has been made in consideration of such problems existing in prior art, and an object thereof is to acquire highly accurate object information in an apparatus which processes a photoacoustic signal generated due to light irradiated via a replaceable optical member.

First aspect of the disclosure is an information processing apparatus which processes image data indicating characteristic information at a plurality of positions inside an object based on an acoustic wave generated when the object is irradiated with light via a light irradiating unit, the positional relationship of which with an acoustic probe is defined, the information processing apparatus comprising:

an image data acquiring unit configured to acquire the image data;

an information acquiring unit configured to acquire identification information which identifies a type of the acoustic probe; and

a correcting unit configured to correct, based on the identification information, an effect on an image value of the image data attributable to a variation in light intensities, at the plurality of positions inside the object, of light irradiated via the light irradiating unit.

Second aspect of the disclosure is a system, comprising:

a light irradiating unit configured to irradiate an object with light;

an acoustic probe configured to convert an acoustic wave generated by light irradiation to the object into an electrical signal;

a mounting part configured to mount at least one of the light irradiating unit and the acoustic probe so as to define a positional relationship between the light irradiating unit and the acoustic probe; and

an information processing apparatus configured to generate image data indicating characteristic information at a plurality of positions inside the object based on the electrical signal, wherein

the information processing apparatus includes:

an information acquiring unit configured to acquire identification information which identifies a type of the acoustic probe; and

a correcting unit configured to correct, based on the identification information, an effect on an image value of the image data attributable to a variation in light intensities, at the plurality of positions inside the object, of light irradiated via the light irradiating unit.

Third aspect of the disclosure is an information processing apparatus which processes image data indicating characteristic information at a plurality of positions inside an object based on an acoustic wave generated when the object is irradiated with light via a light irradiating unit, the positional relationship of which with an acoustic probe is defined, the information processing apparatus comprising:

an image data acquiring unit configured to acquire the image data;

an information acquiring unit configured to acquire irradiating unit information which is information related to the light irradiating unit; and

a correcting unit configured to correct, based on the irradiating unit information, an effect on an image value of the image data attributable to a variation in light intensities, at the plurality of positions inside the object, of light irradiated via the light irradiating unit.

Fourth aspect of the disclosure is a system, comprising:

a light irradiating unit configured to irradiate an object with light;

an acoustic probe configured to convert an acoustic wave generated by light irradiation to the object into an electrical signal;

a mounting part configured to mount at least one of the light irradiating unit and the acoustic probe so as to define a positional relationship between the light irradiating unit and the acoustic probe; and

an information processing apparatus configured to generate image data indicating characteristic information at a plurality of positions inside the object based on the electrical signal, wherein

the information processing apparatus includes:

an information acquiring unit configured to acquire irradiating unit information which is information related to the light irradiating unit; and

a correcting unit configured to correct, based on the irradiating unit information, an effect on an image value of the image data attributable to a variation in light intensities, at the plurality of positions inside the object, of light irradiated via the light irradiating unit.

Further features of the present invention will become apparent from the following description of exemplary embodiments (with reference to the attached drawings).

### BRIEF DESCRIPTION OF THE DRAWINGS

FIG. 1 is a configuration diagram of a photoacoustic apparatus according to an embodiment;

FIGS. 2A and 2B are perspective views showing a probe according to an embodiment;

FIG. 3 is a flow chart of processes performed by a photoacoustic apparatus according to an embodiment;

FIGS. 4A and 4B are diagrams schematically showing an effect of an embodiment; and

FIG. 5 represents an example of a display screen output via a display apparatus.

### DESCRIPTION OF THE EMBODIMENTS

Hereinafter, preferred embodiments of the present invention will be described with reference to the drawings. However, it is to be understood that dimensions, materials, shapes, relative arrangements, and the like of components described below are intended to be changed as deemed appropriate in accordance with configurations and various conditions of apparatuses to which the present invention is to be applied. Therefore, the scope of the present invention is not intended to be limited to the embodiments described below.

The present invention relates to a technique for detecting an acoustic wave propagating from an object and generating and acquiring characteristic information of the inside of the object. Accordingly, the present invention can also be considered an object information acquiring apparatus or a control method thereof, or an object information acquiring method. In addition, the present invention can also be considered an information processing apparatus which generates object information based on an acoustic wave or a control method thereof, or an information processing method.

The present invention can also be considered a program that causes an information processing apparatus including hardware resources such as a CPU and a memory to execute these methods or a computer-readable non-transitory storage medium storing the program.

The information processing apparatus according to the present invention is an apparatus which processes a signal obtained by receiving an acoustic wave generated inside an object when the object is irradiated with light (an electromagnetic wave) and which acquires characteristic information of the object as image data. In this case, characteristic information refers to information on a characteristic value which corresponds to each of a plurality of positions inside the object and which is generated using a received signal obtained by receiving a photoacoustic wave.

Characteristic information acquired by photoacoustic imaging is a value reflecting an absorption rate of light energy. For example, characteristic information includes a generation source of an acoustic wave generated by light irradiation, initial sound pressure inside an object, a light energy absorption density or an absorption coefficient derived from initial sound pressure, or a concentration of substances constituting tissue.

In addition, a distribution of oxygen saturation can be calculated by obtaining a concentration of oxygenated hemoglobin and a concentration of reduced hemoglobin as concentrations of substances. Furthermore, a glucose concentration, a collagen concentration, a melanin concentration, a volume fraction of fat or water, and the like can also be obtained. Moreover, substances with a characteristic light absorption spectrum including contrast agents administered into the body may also be considered subjects of characteristic information acquisition.

The information processing apparatus according to the present invention includes an apparatus which processes a signal obtained by receiving an ultrasonic wave (an ultrasonic echo) reflected inside an object and which acquires characteristic information of the object as image data. In this case, the acquired characteristic information is information reflecting a difference in acoustic impedances in tissue inside the object.

A two-dimensional or three-dimensional characteristic information distribution is obtained based on characteristic information at each position inside the object. Distribution data may be generated as image data. Characteristic information may be obtained as distribution information of respective positions inside the object instead of as numerical data. In other words, distribution information such as an initial sound pressure distribution, an energy absorption density distribution, an absorption coefficient distribution, and an oxygen saturation distribution can be obtained. Image data refers to data indicating characteristic information at a plurality of positions inside the object.

An acoustic wave in the present specification is typically an ultrasonic wave and includes an elastic wave which is also referred to as a sonic wave or an acoustic wave. An electrical signal transformed from an acoustic wave by a probe or the like is also referred to as an acoustic signal. However, descriptions of an ultrasonic wave and an acoustic wave in the present specification are not intended to limit a wavelength of the elastic waves. An acoustic wave generated by a photoacoustic effect is referred to as a photoacoustic wave or an optical ultrasonic wave. An electrical signal derived from a photoacoustic wave is also referred to as a photoacoustic signal. It should be noted that, in the present specification, a photoacoustic signal is a concept encompassing both analog signals and digital signals. Distribution data is also referred to as photoacoustic image data or reconstructed image data.

### Description of Embodiment

### Apparatus Configuration

Hereinafter, a preferred embodiment of the present invention will be described in detail with reference to the drawings.

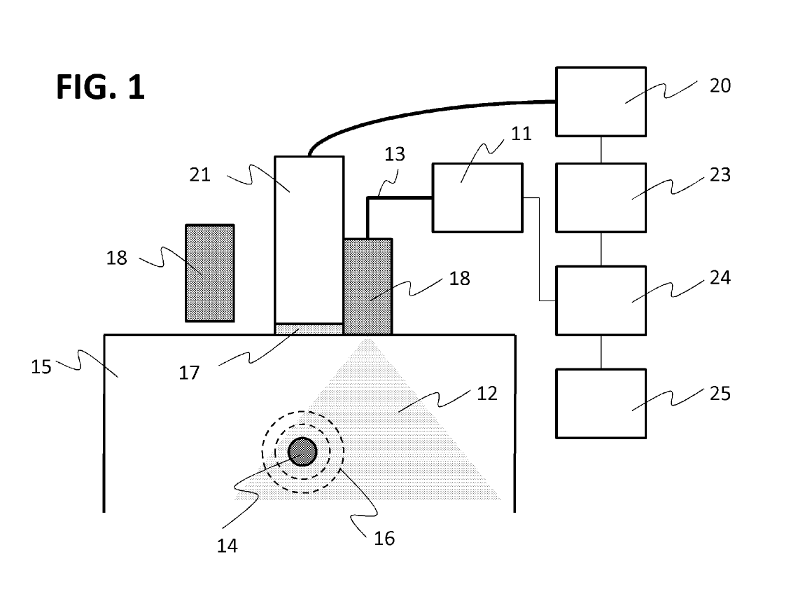

FIG. 1 is a diagram illustrating a configuration of a photoacoustic apparatus (system) according to the present embodiment. The photoacoustic apparatus (system) according to the present embodiment is configured so as to have an acoustic probe 21 including an acoustic element 17, a light source 11, an optical system 13, a light irradiating unit 18 attachable to and detachable from the acoustic probe 21, an information processing unit 23, and a display controlling unit 24. The information processing unit 23 and the display controlling unit 24 may be collectively considered an information processing apparatus. In other words, the information processing apparatus according to the present invention may be described as being provided with the information processing unit 23 and the display controlling unit 24.

An outline of photoacoustic imaging with respect to an object will now be described.

Light emitted from the light source 11 is guided to the light irradiating unit 18 via the optical system 13 including a light transmission path constituted by an optical fiber or the like, processed so as to attain desired characteristics by an optical system such as a lens or a diffuser plate built into the light irradiating unit 18, and finally irradiated to an object 15.

When a part of energy of the light 12 having propagated inside the object 15 is absorbed by a light absorber 14 such as a blood vessel, an acoustic wave 16 (typically, an ultrasonic wave) is generated due to thermal expansion. The acoustic wave 16 is detected by the acoustic element 17 inside the acoustic probe, and amplified and digitally converted by a signal acquiring unit 20. The converted signal is converted into photoacoustic image data representing characteristics of the object by the information processing unit 23 and the display controlling unit 24, and displayed as an image on a display apparatus 25.

Next, details of each component will be described.

### Light Source 11

The light source 11 is an apparatus that generates pulse light for irradiating an object. While the light source may be a laser light source in order to obtain a large output, a light-emitting diode, a flash lamp, a microwave source, or the like may be used in place of a laser. Moreover, when using an electromagnetic wave, such as a microwave and a radio wave having a different wavelength from light, the light source can also be referred to as an electromagnetic wave source.

When using a laser as the light source, various lasers such as a solid-state laser, a gas laser, a dye laser, and a semiconductor laser can be used. For example, a pulse laser such as a Nd:YAG laser or an alexandrite laser may be used as the light source. In addition, a Ti:sa laser using Nd:YAG laser light as excitation light or an optical parametric oscillator (OPO) laser may be used as the light source.

In addition, a wavelength of the pulse light may be a specific wavelength which is absorbed by a specific component among the components constituting the object and which enables light to propagate to the inside of the object. Specifically, when the object is a living organism, the wavelength may be at least 500 nm and not more than 1400 nm.

Furthermore, in order to effectively generate a photoacoustic wave, light must be irradiated in a sufficiently short period of time in accordance with thermal characteristics of the object. When the object is a living organism, a pulse width of the pulse light generated by the light source can range from 1 to 1000 nanoseconds.

### Optical System 13

Light emitted from the light source 11 is propagated via the optical system 13 and irradiated on a surface of the object. An optical element such as a mirror and an optical fiber can be used as the optical system 13. The optical system 13 may be configured using a diffuser plate, a lens, or the like in order to give an irradiation pattern of pulsed light a desired shape. Moreover, the optical system 13 may be built into the light irradiating unit 18 to be described later.

### Object 15 (Light Absorber 14)

Although the object 15 does not constitute the present invention, a description thereof will be given below. The photoacoustic apparatus according to the present embodiment can be used for the purposes of diagnosing a malignant tumor, a vascular disease, and the like, performing a follow-up observation of chemotherapy, and the like of a human or an animal. Therefore, as the object 15, a diagnostic subject site such as a living organism or, more specifically, breasts, respective internal organs, the vascular network, the head, the neck, the abdominal area, and the extremities including fingers and toes of a human or an animal is assumed. For example, when the measurement subject is a human body, a subject of a light absorber may be oxyhemoglobin, deoxyhemoglobin, a blood vessel containing oxyhemoglobin or deoxyhemoglobin in a large amount, or a new blood vessel formed in a vicinity of a tumor. In addition, the subject of a light absorber may be a plaque on a carotid artery wall or the like. Furthermore, pigments such as methylene blue (MB) and indocyanine green (ICG), gold particulates, or an externally introduced substance which is an accumulation of or which is chemically modified with such pigments or gold particulates may be used as a light absorber.

### Acoustic Element 17

The acoustic element 17 is a unit which detects an acoustic wave and converts the acoustic wave into an analog electrical signal. An acoustic probe is also referred to as a probe, an acoustic element, an acoustic wave detecting element, an acoustic wave detector, an acoustic wave receiver, and a transducer. Since an acoustic wave generated by a living organism is an ultrasonic wave ranging from 100 kHz to 100 MHz, an element capable of receiving this frequency band is used as the acoustic element 17. Specifically, a transducer using a piezoelectric phenomenon, a transducer using optical resonance, a transducer using a variation in capacity, or the like can be used.

Moreover, when the photoacoustic apparatus according to the present embodiment has a function of performing ultrasonic imaging, the acoustic element 17 may have a function of transmitting an acoustic wave formed on an arbitrary wavefront.

An element with high sensitivity and a wide frequency band may be used as the acoustic element. Specific examples of the acoustic element include a piezoelectric element using lead zirconate titanate (PZT) or the like, a polymer piezoelectric film material such as polyvinylidene fluoride (PVDF), a capacitive micromachined ultrasonic transducer (CMUT), and an acoustic element using a Fabry-Perot interferometer. However, the acoustic element is not limited to these examples and any acoustic element may be used as long as functions as a probe are satisfied.

As the acoustic element 17, a plurality of elements that transmit and receive acoustic waves may be arranged in an array or a single element may be adopted. Moreover, the plurality of elements may be arranged in any shape. In particular, based on the principles of image reconstruction, the plurality of elements are favorably arranged in a planar shape, a columnar shape, or a spherical shape, or in a part thereof. The example shown in FIG. 1 represents a one-dimensional or two-dimensional arrangement of the plurality of acoustic elements 17.

The use of such multi-dimensionally arranged acoustic wave detecting elements enables acoustic waves to be simultaneously detected at a plurality of locations and measurement time to be shortened. As a result, the effects of swaying of the object and the like can be reduced. However, when acoustic elements are scannable, an array arrangement need not necessarily be adopted. In addition, when measuring only a change in acoustic signals at a specific location or, in other words, when imaging is not required, only one acoustic element is required and scanning need not be performed.

Moreover, scanning may be further performed by acoustic elements in a multi-dimensional arrangement in order to detect acoustic waves at a greater variety of positions.

### Acoustic Probe 21

The acoustic probe 21 is a probe which has a grip part to be gripped by an operator and which houses the acoustic element 17. The grip part also functions as a housing which holds components of the probe. The grip part may have a cylindrical shape, an elliptic cylindrical shape, a rectangular columnar shape, and the like. In addition, irregularities may be provided so as to give the operator a better grip. An acoustic wave detection surface is provided inside the acoustic probe 21, and at least one acoustic element 17 (transducer) is provided on the detection surface. Furthermore, the acoustic probe 21 is attachable/detachable and replaceable with the signal acquiring unit 20 (to be described later) and, depending on the purpose of measurement, an acoustic probe having various characteristics (number of elements and frequency) and shapes (linear, sectoral, three-dimensional, and the like) can be selected from a plurality of types. Basically, acoustic probes with characteristics and shapes usable in ordinary ultrasonic apparatuses are also usable in the present invention.

### Light Irradiating Unit 18

The light irradiating unit 18 is a unit which is mountable to and detachable from the acoustic probe 21 and which irradiates the object 15 with light propagated via the optical system 13. The light irradiating unit 18 may include an optical system constituted by a diffuser plate, a lens, a prism, or the like which processes the propagated light into a desired shape. In addition, when the light source 11 has a small size as in the case of a semiconductor laser, an LED, and the like, the light source 11 may be built into the light irradiating unit 18.

In the present embodiment, the light irradiating unit 18 can be mounted to and detached from acoustic probes 21 of various shapes.

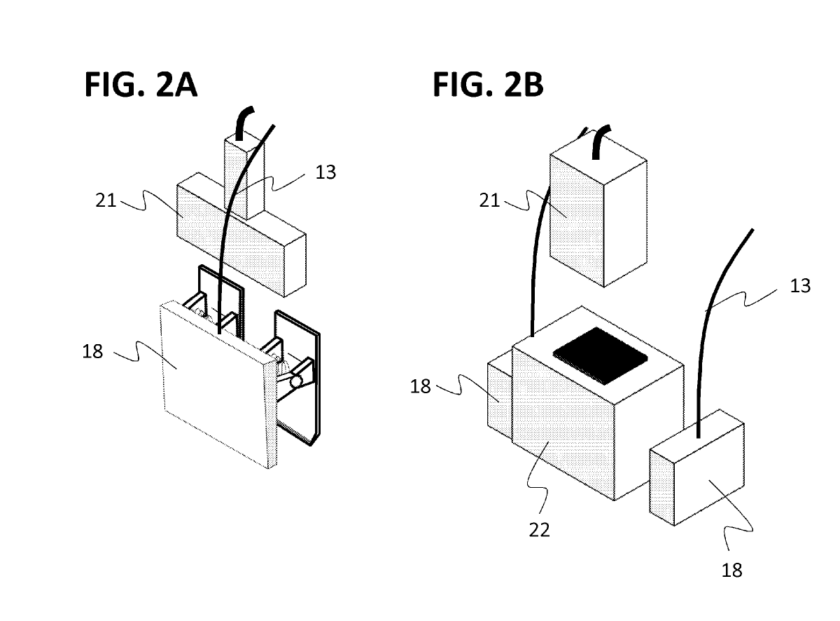

FIG. 2 is a schematic view showing an example of the light irradiating unit 18 which is mountable to and detachable from the acoustic probe 21. For example, as shown in FIG. 2A, by integrating the light irradiating unit with a grip, the light irradiating unit can be attached to acoustic probes 21 with different shapes. In addition, as shown in FIG. 2B, a dedicated mounting part 22 may be provided for each acoustic probe 21 and the light irradiating unit 18 may be mounted to the mounting part. Moreover, methods of mounting and detaching the light irradiating unit 18 to and from the acoustic probe 21 are not limited to the methods shown in FIG. 2 and any method may be adopted such as fixing by a screw or a pin and using a fastener. By mounting at least one of the light irradiating unit 18 and the acoustic probe 21 to the mounting part 22, a positional relationship between the light irradiating unit 18 and the acoustic probe 21 is defined. The clip shown in FIG. 2A also corresponds to a mounting part. The mounting part for defining the positional relationship between the light irradiating unit and the acoustic probe may be provided on the light irradiating unit or on the acoustic probe. Alternatively, the mounting part may be constructed independently of the light irradiating unit and the acoustic probe. In other words, as long as the positional relationship between the light irradiating unit and the acoustic probe can be defined, the mounting part need only be capable of being mounted with at least one of the light irradiating unit and the acoustic probe. In addition, the mounting part may be capable of being replaceably mounted with a plurality of types of light irradiating units. Furthermore, the mounting part may be capable of being replaceably mounted with a plurality of types of acoustic probes.

### Signal Acquiring Unit 20

The signal acquiring unit 20 is a unit which amplifies an analog electrical signal obtained by the acoustic element 17 and converts the amplified electrical signal into a digital signal. The signal acquiring unit 20 is typically constituted by an amplifier, an A/D converter, a field programmable gate array (FPGA) chip, or the like. When a plurality of signals are obtained from the acoustic probe, a plurality of signals may be processed simultaneously. Accordingly, a period of time until an image is formed can be reduced. Moreover, a “photoacoustic signal” as used in the present specification is a concept including both an analog signal acquired from the acoustic element 17 and a digital signal obtained by subsequent A/D conversion. Moreover, when the acoustic element 17 has a function of transmitting an ultrasonic wave to an object, an electric circuit for transmitting an ultrasonic wave via the acoustic element may be built into the signal acquiring unit 20.

### Information Processing Unit 23

The information processing unit 23 is a unit which converts a digital signal obtained by the signal acquiring unit 20 into image data (photoacoustic image data) representing characteristic information of an object. Generally, characteristic information of an object refers to an initial sound pressure distribution, a distribution of light absorption energy density, or information related thereto.

The information processing unit 23 applies an image reconstruction algorithm generally used in photoacoustic imaging to the digital signal acquired by the signal acquiring unit 20 and acquires information (photoacoustic image data) related to optical characteristics of the inside of the object. As the image reconstruction algorithm, while time-domain back-projection is generally used, a method such as an inverse problem analysis method by iterative processing can also be used. Other representative image reconstruction methods include time-domain reconstruction methods such as universal back-projection and filtered back-projection.

On the other hand, when ultrasonic imaging is used in combination with photoacoustic imaging, a beam forming technique (a phasing addition process) generally used in ultrasonic imaging apparatuses is applied to a signal obtained when receiving a reflected wave (an ultrasonic echo) reflected by the object, and object information is acquired as image data.

Moreover, when performing photoacoustic imaging, object information can be formed without image reconstruction by using a focused acoustic probe. In such a case, signal processing using an image reconstruction algorithm need not be performed.

A general-purpose computer, a dedicated electronic circuit board, or the like is used as the information processing unit 23. Moreover, the signal acquiring unit 20 and the information processing unit 23 may be integrated with each other. In this case, image data of an object can also be generated by hardware processing instead of software processing such as that performed by a computer.

### Display Controlling Unit 24

The display controlling unit 24 as an information acquiring unit acquires information related to the light irradiating unit 18. In addition, based on the information related to the light irradiating unit 18, the display controlling unit 24 as a correcting unit corrects photoacoustic image data (hereinafter, first photoacoustic image data) generated by the information processing unit 23 and generates image data (hereinafter, second photoacoustic image data) to be provided to a user (an operator).

Specifically, the second photoacoustic image data is generated by performing image processing based on photoacoustic image data generated by the information processing unit 23 and information related to the light irradiating unit 18 mounted to the acoustic probe. For example, image processing includes, but is not limited to, brightness conversion, clipping of a region of interest, an extraction process of a blood vessel, a separation process of arteries and veins, and a logarithmic compression process in accordance with a light intensity distribution inside the object.

Moreover, while “information related to a light irradiating unit” as described herein refers to information for calculating a light intensity distribution (a light fluence distribution) inside the object and is typically identification information which indicates an identifier for identifying an individual light irradiating unit or a type thereof, other information may be used. For example, the information related to a light irradiating unit may be information identifying a type of a probe mounted with the light irradiating unit, a type or a shape of a mounting part which connects the light irradiating unit and the probe with each other, a shape of a region (hereinafter, a light irradiation region) in which light is irradiated onto the object, and whether or not the light irradiating unit is mounted.

Hereinafter, the information related to a light irradiating unit will be referred to as irradiating unit information. Using irradiating unit information enables a difference in light intensity distribution which is created for each light irradiating unit type to be corrected.

Furthermore, the display controlling unit 24 as a display controlling unit generates information to accompany object information and outputs the information at the same time as an image representing the object information. For example, a mounted/detached state of the light irradiating unit, a type of the light irradiating unit being used, a wavelength of laser light, or an image or a display representing a light irradiation position is output together with first or second photoacoustic image data.

Moreover, the display controlling unit 24 may temporarily record the first photoacoustic image data and irradiating unit information, and generate the second photoacoustic image data by post-processing.

In addition, the display controlling unit 24 may output image data (for example, ultrasonic image data) other than photoacoustic image data. Furthermore, in this case, additional image processing may be performed.

### Display Apparatus 25

The display apparatus 25 is an apparatus for displaying an image output by the display controlling unit 24 and, typically, a liquid crystal display or the like is used. Moreover, the display apparatus 25 may be provided separate from the photoacoustic apparatus according to the present embodiment.

### Flow Chart of Processes

Next, processes performed by the photoacoustic apparatus according to the present embodiment will be described. In the following processes, the display controlling unit 24 acquires irradiating unit information and, based on the information, the display controlling unit 24 performs correction of the first photoacoustic image data generated by the information processing unit 23.

Moreover, it is assumed that the processes described below are started in a state where a photoacoustic signal attributable to light irradiated onto the object is stored in a memory (not shown) in the apparatus and are executed by the information processing unit 23 or the display controlling unit 24.

First, in step 5301, the information processing unit 23 acquires a photoacoustic signal digitally-converted by the signal acquiring unit 20 and temporarily stores the photoacoustic signal.

Next, in step S302, using the stored photoacoustic signal, the information processing unit 23 generates first photoacoustic image data representing a distribution of optical characteristic values inside the object. For example, image data representing an initial sound pressure distribution, a distribution of light absorption energy density, or a distribution related thereto is generated as the first photoacoustic image data. The image data is data dependent on a method of light irradiation.

Next, in step S303, the display controlling unit 24 acquires irradiating unit information.

While irradiating unit information is typically identification information which indicates an identifier for identifying an individual light irradiating unit or a type thereof, other information may be used. For example, the irradiating unit information may be information identifying a type of a probe mounted with the light irradiating unit, a type or a shape of a mounting part which connects the light irradiating unit and the probe with each other, a shape of a light irradiation region, whether or not the light irradiating unit is mounted, or information related to a wavelength or an intensity of light.

The irradiating unit information is information necessary for calculating a light intensity distribution of light reaching a plurality of positions inside the object. As the information, for example, information electrically recorded in the acoustic probe 21 may be acquired via the signal acquiring unit 20. Alternatively, information electrically recorded in the light irradiating unit 18 may be acquired via a control circuit inside the light source 11. Alternatively, information electrically recorded in the light irradiating unit 18 may be acquired by the information processing unit 23 or the display controlling unit 24 in a wireless manner. As described above, the information may be acquired using any method regardless of whether the method is wired or wireless.

Next, in step 5304, the display controlling unit 24 performs image processing on the first photoacoustic image data based on the acquired irradiating unit information.

As described earlier, the first photoacoustic image data is data representing an initial sound pressure distribution, a distribution of light absorption energy density, or the like inside the object. A distribution of light absorption energy density will now be described as an example.

As expressed by Expression (1), a distribution of light absorption energy density H(r) is proportional to an absorption coefficient distribution μₐ(r) inside the object and a light intensity Φ(r) which reaches inside the object.

H(r)∝μₐ(r)·Φ(r) (1)

As is apparent from Expression (1), H(r) is greatly affected by the light intensity OW which reaches inside the object. Light irradiated on an object that is a living organism is disseminated and absorbed, and is damped significantly as a propagation distance of the light increases. 1.74 cm⁻¹ which is an equivalent damping coefficient of a general living organism indicates that, when light propagates 1.74 cm inside the living organism, energy decreases to at least 37% or less. In other words, since the light intensity decreases exponentially in accordance with a distance from the light irradiating unit, brightness of the first photoacoustic image data varies significantly depending on location. That is, a dynamic range to be displayed widens. On the other hand, when the dynamic range widens, visibility significantly declines and the likelihood of overlooking a light absorber with low brightness increases.

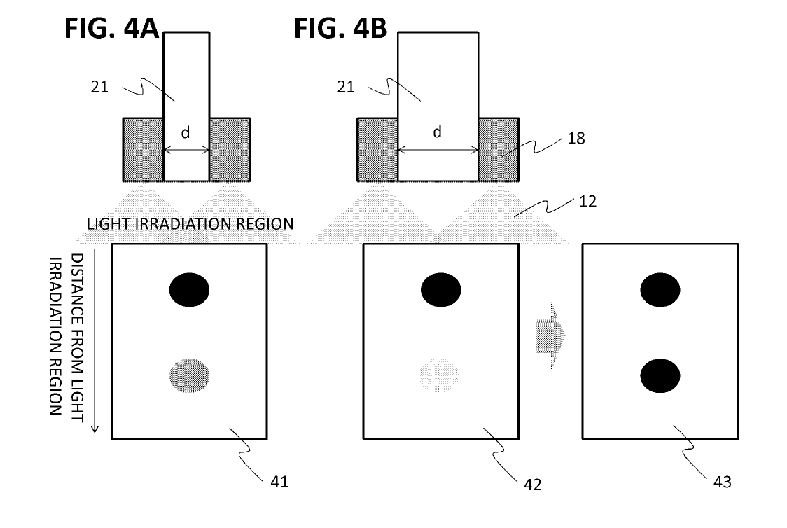

A specific example is shown in FIG. 4. FIG. 4 is a diagram showing a situation in which two light irradiating units are mounted to both sides of the acoustic probe 21. As shown, a spacing d between the two light irradiating units differs depending on a shape of the acoustic probe 21.

Reference numeral 41 denotes an example of first photoacoustic image data obtained using an acoustic probe shown in FIG. 4A, and reference numeral 42 denotes an example of first photoacoustic image data obtained using an acoustic probe shown in FIG. 4B.

In the first photoacoustic image data, the further away from a light irradiation region, the lower the brightness of a pixel. In the present example, since the spacing between the light irradiating units is narrower in FIG. 4A than in FIG. 4B, a light intensity which reaches a deep part of the object is smaller and brightness on an image is lower in the case of FIG. 4B. While FIG. 4B is illustrated as though a light intensity distribution varies depending on depth, even at a same depth, a difference in light intensities is created when horizontal positions differ.

In this manner, since a distribution of light reaching the inside of the object varies depending on an arrangement or a type of light irradiating units, the visibility of a light absorber changes. In the present embodiment, in order to solve this problem, the first photoacoustic image data is corrected using irradiating unit information.

Generally, a light intensity distribution inside an object (a variation in light intensities at a plurality of positions inside the object) can be calculated by solving a light diffusion equation (or a transport equation) provided below.

∇·(D(r)∇φ(r))−(vμₐ(r))φ(r)=−vS(r) (2)

In Expression (2), D(r) denotes a diffusion coefficient distribution, ϕ(r) denotes a light intensity distribution inside the object, v denotes the speed of light, μₐ(r) denotes an absorption coefficient distribution, S(r) denotes a distribution of irradiation light, and r denotes a three-dimensional position inside the object. Since an average diffusion coefficient and an average absorption coefficient are determined once a measurement site of the object is decided, the light intensity distribution inside the object can be calculated if the distribution of irradiation light denoted by S(r) is known.

As is apparent from FIG. 4, the light intensity distribution of irradiation light can be estimated based on a type (shape) of the probe to which the light irradiating unit is mounted and on a position at which the light irradiating unit is mounted. In other words, once a relative positional relationship between the acoustic probe and the light irradiating unit is determined, the light intensity distribution inside the object can be estimated and a correction with respect to the first photoacoustic image data can be performed.

A relative positional relationship between the acoustic probe and the light irradiating unit can be determined by acquiring the irradiating unit information described earlier.

For example, by acquiring a type of the acoustic probe and a type of the light irradiating unit, a position of the object to be irradiated with light can be specified and, as a result, the light intensity distribution inside the object can be calculated as described above. Moreover, the light intensity distribution can be stored in association with irradiating unit information or may be calculated whenever necessary. In other words, the display controlling unit 24 may be provided with a memory as a storage unit in which a plurality of pieces of irradiating unit information (identification information or the like) and information indicating a variation in light intensities at a plurality of positions inside the object are associated with each other. In addition, the display controlling unit 24 may read out, from the memory, information indicating a variation in light intensities at a plurality of positions inside the object which corresponds to the irradiating unit information acquired in step S303, and use the information in a correction process of correcting the variation in light intensities.

As a result, for example, by dividing the first photoacoustic image data denoted by reference numerals 41 and 42 by the calculated light intensity distribution, photoacoustic image data after correction (second photoacoustic image data) such as that denoted by reference numeral 43, in which the influence of the light intensity distribution is reduced, can be obtained. In other words, the present correction process enables a variation in image values of the first photoacoustic image data attributable to a variation in light intensities at a plurality of positions inside the object to be corrected. Accordingly, regardless of the shape of an acoustic probe to which a light irradiating unit is mounted, a user can be provided with image data with high visibility.

Moreover, while the described present example is an example in which a same light irradiating unit is mounted to acoustic probes of different shapes, a similar effect can be produced even when mounting light irradiating units with different characteristics to acoustic probes having a same shape. In the former case, identification information of the acoustic probe may be adopted as irradiating unit information, and in the latter case, identification information of the light irradiating unit may be adopted as irradiating unit information. It is needless to say that, when both are equally replaceable, both the identification information of the light irradiating unit and the identification information of the acoustic probe may be adopted as irradiating unit information.

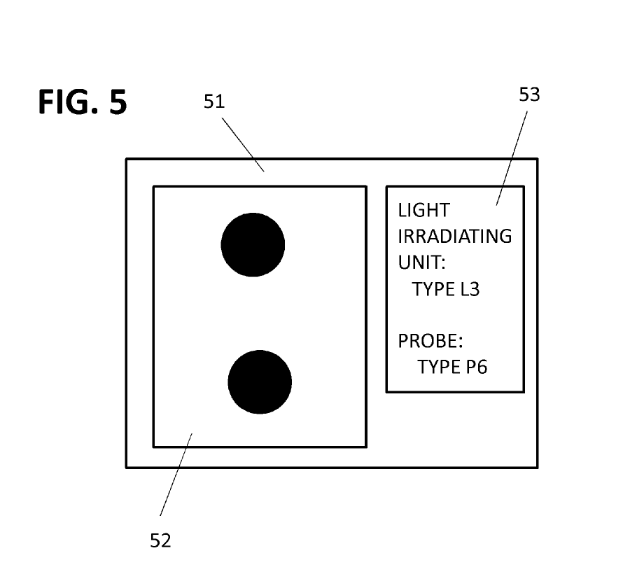

Next, in step S305, as shown in FIG. 5, the display controlling unit 24 causes various display items related to the irradiating unit information to be displayed side by side with the first or second photoacoustic image data on a liquid crystal display that is the display apparatus 25. In the present example, a type of the light irradiating unit and a type of the acoustic probe are displayed together with the photoacoustic image data after correction (the second photoacoustic image data).

Moreover, the information to be displayed is not limited to the above as long as the information is related to the light irradiating unit. For example, a type and a shape of the light irradiating unit, a type and a shape of the acoustic probe to which the light irradiating unit is mounted, a type and a shape of a mounting part, a shape of a light irradiation region, whether or not the light irradiating unit is mounted, or a light irradiation position with respect to the object may be displayed. In addition, when the light irradiating unit has a light source, a wavelength of light, an irradiated light intensity, or the like may be displayed. Moreover, irradiating unit information need not necessarily be displayed.

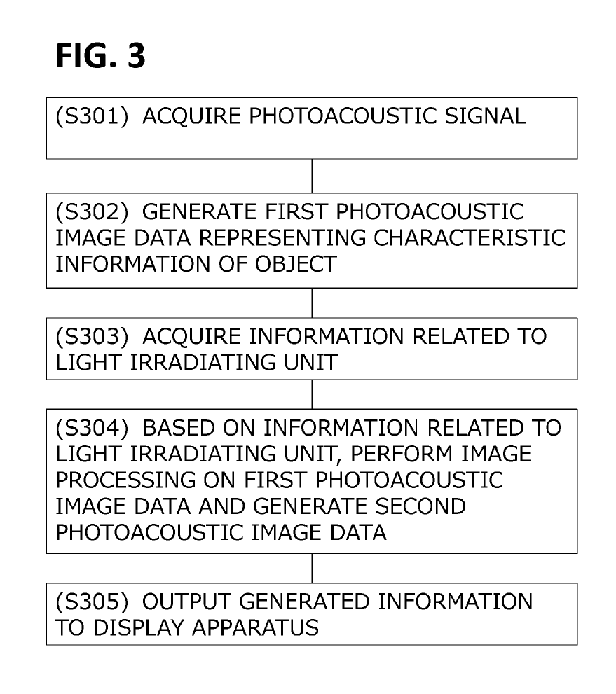

Moreover, while the second photoacoustic image data is generated in step 5304 and displayed in step 5305 in the example shown in FIG. 3, irradiating unit information may be saved together with the first photoacoustic image data, and the second photoacoustic image data may be generated by post-processing.

In this case, image data may be saved using a format (for example, the DICOM format) which enables information related to measurement to be retained separately from an image. For example, an image can be saved in a state where irradiating unit information is stored in a header.

In addition, accordingly, a correction process of image data can be performed using image processing software or the like installed on another computer or the like. It is needless to say that the processes of steps S304 and S305 may be performed after saving information.

### First Practical Example

Next, an example of a photoacoustic apparatus to which the embodiment described above has been applied will be described.

In the present practical example, a Ti:sa laser system excited by a second harmonic YAG laser is used as the light source 11. A Ti:sa laser is capable of irradiating an object with light with a wavelength of 700 to 900 nm. Moreover, laser light is guided to the light irradiating unit 18 incorporating an expansion optical system using an optical fiber which is a light transmission path. In addition, the laser light is expanded to 5×20 mm at the light irradiating unit. Furthermore, the light irradiating unit is mounted to an acoustic probe (15 mm×50 mm) used in an ordinary ultrasonic diagnostic apparatus. The acoustic probe is a linear probe with a central frequency of 7.5 MHz.

The light irradiating unit is mounted with a clip as shown in FIG. 2A so as to be mountable to a probe in any shape. In addition, the acoustic probe is connected to the signal acquiring unit 20 and is capable of receiving data in synchronization with light irradiation.

The object 15 is a rectangular parallelepiped phantom simulating a living organism and includes urethane rubber mixed with titanium oxide as a scatterer and ink as an absorber. In addition, a wire which strongly absorbs light is embedded as the light absorber 14 inside the rectangular parallelepiped phantom.

The phantom is irradiated with light with a wavelength of 756 nm from the Ti:sa laser, and an obtained analog detected signal is saved in a memory inside a computer that is the information processing unit 23 as a digital signal. Next, using the digital detected signal, image reconstruction is performed with software corresponding to the information processing unit 23. A time-domain UBP method is used as the method of image reconstruction. Accordingly, first photoacoustic image data which is a distribution of light absorption energy density is obtained.

Moreover, in the first photoacoustic image data, due to damping of light inside the phantom, brightness of a wire target separated from a light irradiation region is significantly lower than brightness of a wire target near the light irradiation region.

Next, using software corresponding to the display controlling unit 24 which is installed in the same computer as the information processing unit 23, image processing is performed on the first photoacoustic image data in order to correct the first photoacoustic image data to an image with higher visibility.

First, the computer acquires an ID (identification information) of the acoustic probe through the signal acquiring unit 20.

In the first practical example, a common light irradiating unit is used, and the light intensity distribution inside the object varies depending on a type of the acoustic probe to which the light irradiating unit is mounted. Therefore, the ID of the acoustic probe is used as irradiating unit information. In the present practical example, the ID, a light irradiation position, and a positional relationship with a detecting element are recorded in advance in a table (LUT) format in a memory inside the computer, and a rough light intensity distribution inside the object is estimated using the LUT data.

Subsequently, the first photoacoustic image is divided by the estimated light intensity distribution to generate second photoacoustic image data after correction. As a result, second photoacoustic image data with high visibility such as that denoted by reference numeral 43 was obtained in which a variation in brightness of the target had been reduced.

In addition, even when the acoustic probe was replaced with a convex-type acoustic probe with a different shape, a similar process was performed based on the ID (identification information) of the acoustic probe and a similar image with high visibility was obtained.

As described above, according to the first practical example, it was confirmed that a photoacoustic image with high visibility can be generated even when mounting an attachable and detachable light irradiating unit to various hand-held probes with different shapes.

### Second Practical Example

In a second practical example, an alexandrite laser which is a solid state laser and which generates light with a wavelength of 755 nm is used as the light source 11. In addition, a urethane phantom simulating a breast shape is used as the phantom. A light absorber similar to that of the first practical example is arranged inside the phantom.

Furthermore, in the present practical example, a linear probe (outer shape: 50 mm×50 mm) for three-dimensional images is used as the acoustic probe 21. Oscillated laser light is guided through a bundle-type optical fiber to a light irradiating unit that can be connected to the acoustic probe. The light irradiating unit is configured to be capable of irradiating light from a plurality of locations by bifurcating a plurality of fibers.

As shown in FIG. 1, the light irradiating unit is connected to the linear probe for three-dimensional images by a screw so as to be capable of irradiating light from two directions.

The second practical example is a mode in which both the light irradiating unit and the acoustic probe are replaceable. Therefore, both the ID of the light irradiating unit and the ID of the acoustic probe are used as irradiating unit information.

The light irradiating unit has a built-in wireless IC tag and is configured to be capable of wirelessly transmitting the ID of the light irradiating unit to the computer which is an information processing unit and a display controlling unit. In addition, a signal acquiring unit is configured to be capable of wirelessly transmitting the ID of the acoustic probe to the computer which is the information processing unit and the display controlling unit.

In the second practical example, the phantom is irradiated with light with a wavelength of 755 nm from the alexandrite laser, and an obtained photoacoustic signal is saved in a memory inside a computer that is the information processing unit 23. Next, using the photoacoustic signal, image reconstruction is performed with software corresponding to the information processing unit 23. A model-based reconstruction method is used as the method of image reconstruction. Accordingly, first photoacoustic image data which is an initial sound pressure distribution is obtained.

Next, in a similar manner to the first practical example, image processing is performed on the first photoacoustic image data in order to correct the first photoacoustic image data to an image with higher visibility.

In the present practical example, a computer that is the display controlling unit acquires the ID (identification information) of the light irradiating unit from the light irradiating unit and acquires the ID (identification information) of the acoustic probe from the signal acquiring unit. In addition, based on a positional relationship between an acoustic element and the light irradiating unit acquired based on the respective pieces of identification information, the initial sound pressure distribution that is the first photoacoustic image data is corrected. As a result, second photoacoustic image data with high visibility such as that denoted by reference numeral 43 was obtained in which a variation in brightness of the target had been reduced.

In addition, even when the acoustic probe was replaced with a convex-type acoustic probe with a different shape, a similar process was performed based on the ID (identification information) of the acoustic probe and a similar image with high visibility was obtained.

In the present practical example, in order to make the operator aware of what kind of light irradiating unit is mounted, an attached or detached state of the light irradiating unit and the type of the light irradiating unit being used are output together with the second photoacoustic image data after correction.

As described above, according to the second practical example, it was confirmed that a photoacoustic image with high visibility can be generated even when mounting a replaceable light irradiating unit to various hand-held probes with different shapes.

### Third Practical Example

While a configuration of an apparatus in a third practical example is substantially similar to the second practical example, there is a difference in that the light irradiating unit is connected to a computer that is the display controlling unit via an electric cable and that the light irradiating unit and the acoustic probe are connected by a dedicated mounting part such as that shown in FIG. 2B.

The third practical example is a mode in which the light irradiating unit is replaceable together with the mounting part. Therefore, the ID of the light irradiating unit is used as irradiating unit information.

In the third practical example, using a method similar to that of the second practical example, image reconstruction is performed based on a photoacoustic signal and a distribution of light energy absorption density that is first photoacoustic image data is calculated. In addition, information related to the mounting part is acquired from the light irradiating unit through an electric cable.

Next, a computer that is the information processing unit or the display controlling unit generates and saves a DICOM image which simultaneously records the first photoacoustic image data and information related to the mounting part.

Next, the computer that is the display controlling unit reads information related to the mounting part from the saved DICOM image, and corrects the first photoacoustic image data with a method similar to that of the second practical example. As a result, second photoacoustic image data with high visibility such as that denoted by reference numeral 43 was obtained in which a variation in brightness of the target had been reduced.

In addition, even when the type of the light irradiating unit is changed, a similar process was performed based on the ID (identification information) of the mounting part and a similar image with high visibility was obtained.

In the present practical example, in order to make the user aware of what kind of light irradiating unit is mounted, an approximate irradiation position of light with respect to the object is displayed together with the second photoacoustic image data after correction.

As described above, according to the third practical example, it was confirmed that a photoacoustic image with high visibility can be generated even when mounting various light irradiating units with different characteristics to a same probe.

### Other Embodiments

It is to be understood that the descriptions of the respective embodiments merely present examples of the present invention and, as such, the present invention can be implemented by appropriately modifying or combining the embodiments without departing from the spirit and the scope of the invention.

For example, the present invention may be implemented as an information processing apparatus which performs at least a part of the processes described above. In addition, the present invention may also be implemented as an information processing method which includes at least a part of the processes described above. The present invention may also be implemented as a system (an object information acquiring apparatus) including the information processing apparatus described above or an object information acquiring method which executes the information processing method described above. The processes and units described above may be implemented in any combination thereof insofar as technical contradictions do not arise.

For example, while the display controlling unit 24 performs image correction in the description of the embodiment, image correction may be performed by the information processing unit 23 instead.

In addition, although a mode in which information indicating identifiers of a light irradiating unit and an acoustic probe are electrically read has been cited in the description of the embodiment, for example, image information in which identifiers are encoded may be read optically. The electric/optical information may be retained by the light irradiating unit or the acoustic probe or may be retained by a mounting part or the like which is integrated with the light irradiating unit or the acoustic probe. Alternatively, information indicating an identifier may be acquired by an input operation performed by an operator.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)TM), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-123596, filed on Jun. 23, 2017 and Japanese Patent Application No. 2018-97905, filed on May 22, 2018, which are hereby incorporated by reference herein in their entirety.

What is claimed is:

1. An information processing apparatus which processes image data indicating characteristic information at a plurality of positions inside an object based on an acoustic wave generated when the object is irradiated with light via a light irradiating unit, the positional relationship of which with an acoustic probe is defined, the information processing apparatus comprising:
an image data acquiring unit configured to acquire the image data;
an information acquiring unit configured to acquire identification information which identifies a type of the acoustic probe; and
a correcting unit configured to correct, based on the identification information, an effect on an image value of the image data attributable to a variation in light intensities, at the plurality of positions inside the object, of light irradiated via the light irradiating unit.

2. The information processing apparatus according to claim 1, further comprising a storage unit configured to store, in association with each other, the identification information which identifies the type of the acoustic probe and information indicating the variation in light intensities, at the plurality of positions inside the object, of light irradiated via the light irradiating unit, the positional relationship of which with the acoustic probe is defined,
wherein the correcting unit is configured to (1) read out information indicating the variation in light intensities corresponding to the identification information from the storage unit, and (2) perform a correction process on the image data using the information indicating the variation in light intensities.

3. The information processing apparatus according to claim 1, further comprising a display controlling unit configured to output the identification information and the image data in the form of an image.

4. The information processing apparatus according to claim 1, wherein the information acquiring unit is configured to acquire the identification information electrically stored in the acoustic probe.

5. The information processing apparatus according to claim 1, wherein the information acquiring unit is configured to acquire the identification information by optically reading image information attached to the acoustic probe.

6. The information processing apparatus according to claim 1, wherein the information acquiring unit is configured to acquire the identification information based on an input operation performed by a user.

7. The information processing apparatus according to claim 1, wherein the image data acquiring unit is configured to generate the image data based on an electrical signal converted from the acoustic wave by the acoustic probe.

8. A system, comprising:
a light irradiating unit configured to irradiate an object with light;
an acoustic probe configured to convert an acoustic wave generated by light irradiation to the object into an electrical signal;
a mounting part configured to mount at least one of the light irradiating unit and the acoustic probe so as to define a positional relationship between the light irradiating unit and the acoustic probe; and
an information processing apparatus configured to generate image data indicating characteristic information at a plurality of positions inside the object based on the electrical signal, wherein
the information processing apparatus includes:
an information acquiring unit configured to acquire identification information which identifies a type of the acoustic probe; and
a correcting unit configured to correct, based on the identification information, an effect on an image value of the image data attributable to a variation in light intensities, at the plurality of positions inside the object, of light irradiated via the light irradiating unit.

9. An information processing apparatus which processes image data indicating characteristic information at a plurality of positions inside an object based on an acoustic wave generated when the object is irradiated with light via a light irradiating unit, the positional relationship of which with an acoustic probe is defined, the information processing apparatus comprising:
an image data acquiring unit configured to acquire the image data;
an information acquiring unit configured to acquire irradiating unit information which is information related to the light irradiating unit; and
a correcting unit configured to correct, based on the irradiating unit information, an effect on an image value of the image data attributable to a variation in light intensities, at the plurality of positions inside the object, of light irradiated via the light irradiating unit.

10. The information processing apparatus according to claim 9,
wherein the irradiating unit information includes information related to relative positions of the acoustic probe and the light irradiating unit, and
wherein the correcting unit is configured to correct an effect on an image value of the image data attributable to the variation in light intensities based on the information related to the relative positions of the acoustic probe and the light irradiating unit.

11. The information processing apparatus according to claim 9,
wherein the irradiating unit information includes identification information which identifies a type of the light irradiating unit,
wherein the information processing apparatus further comprises a storage unit configured to store, in association with each other, the identification information and information indicating the variation in light intensities, at the plurality of positions inside the object, of light irradiated to the object via the light irradiating unit, and
wherein the correcting unit is configured to (1) read out information indicating the variation in light intensities corresponding to the identification information from the storage unit, and (2) perform a correction process on the image data using the information indicating the variation in light intensities.

12. The information processing apparatus according to claim 9, wherein the information acquiring unit is configured to acquire the identification information based on an input operation performed by a user.

13. The information processing apparatus according to claim 9, further comprising a display controlling unit configured to output the irradiating unit information and the image data in the form of an image.

14. The information processing apparatus according to claim 9, wherein the information acquiring unit is configured to acquire the irradiating unit information electrically stored in the light irradiating unit.

15. The information processing apparatus according to claim 9, wherein the information acquiring unit is configured to acquire the irradiating unit information by optically reading image information attached to the light irradiating unit.

16. The information processing apparatus according to claim 9, wherein the image data acquiring unit is configured to generate the image data based on an electrical signal converted from the acoustic wave by the acoustic probe.

17. A system, comprising:
a light irradiating unit configured to irradiate an object with light;
an acoustic probe configured to convert an acoustic wave generated by light irradiation to the object into an electrical signal;
a mounting part configured to mount at least one of the light irradiating unit and the acoustic probe so as to define a positional relationship between the light irradiating unit and the acoustic probe; and
an information processing apparatus configured to generate image data indicating characteristic information at a plurality of positions inside the object based on the electrical signal, wherein
the information processing apparatus includes:
an information acquiring unit configured to acquire irradiating unit information which is information related to the light irradiating unit; and
a correcting unit configured to correct, based on the irradiating unit information, an effect on an image value of the image data attributable to a variation in light intensities, at the plurality of positions inside the object, of light irradiated via the light irradiating unit.
